(19) United States
(12) Patent Application Publication
(10) Pub. No.: US 2023/0079014 A1
(43) Pub. Date: Mar. 16, 2023

(54) VALVE IN VALVE SYSTEM AND METHOD

(71) Applicant: BOSTON SCIENTIFIC SCIMED, INC., MAPLE GROVE, MN (US)

(72) Inventor: Michael G. Hayes, Galway (IE)

(73) Assignee: BOSTON SCIENTIFIC SCIMED, INC., MAPLE GROVE, MN (US)

(21) Appl. No.: 17/941,229

(22) Filed: Sep. 9, 2022

Related U.S. Application Data
(60) Provisional application No. 63/242,655, filed on Sep. 10, 2021.

(51) Int. Cl. A61F 2/24

(52) U.S. Cl. CPC A61F 2/2412; A61F 2220/0075; A61F 2/2418

(57) ABSTRACT

An implantable heart valve configured for implantation within an existing prosthetic heart valve includes a frame including struts defining a lattice region and a plurality of arches, and a central lumen extending therebetween, a plurality of protrusions extending radially outward from the plurality of struts, and a valve coupled to the frame and positioned within the central lumen.

### CROSS-REFERENCE TO RELATED APPLICATIONS

This application claims the benefit of priority of U.S. Provisional Application No. 63/242,655 filed Sep. 10, 2021, the entire disclosure of which is hereby incorporated by reference.

### TECHNICAL FIELD

The disclosure pertains to medical devices and more particularly to replacement heart valves, and methods for using such medical devices.

### BACKGROUND

A wide variety of medical devices have been developed for medical use including, for example, medical devices utilized to replace heart valves. Heart function can be significantly impaired when a heart valve is not functioning properly. When the heart valve is unable to close properly, the blood within a heart chamber can regurgitate, or leak backwards through the valve. Valve regurgitation may be treated by replacing or repairing a diseased valve, such as an aortic valve. Surgical valve replacement is one method for treating the diseased valve, however this requires invasive surgical openings into the chest cavity and arresting of the patient's heart and cardiopulmonary bypass. Minimally invasive methods of treatment, such as transcatheter aortic valve implantation (TAVI) or transcatheter aortic valve replacement (TAVR), generally involve the use of delivery catheters that are delivered through arterial passageways or other anatomical routes into the heart to replace the diseased valve with an implantable prosthetic heart valve. Leaflets of such valves have been formed from various materials including synthetic materials and animal tissues. Leaflets have been positioned within a frame to maintain or limit the blood flow through a prosthetic valve.

In some cases, many years after implantation, a prosthetic heart valve that was successfully implanted to replace a native valve may itself suffer damage and/or wear and tear. A new prosthetic heart valve may be needed. Of the known medical devices and methods for implanting a new prosthetic heart valve within an existing prosthetic heart valve, each has certain advantages and disadvantages. There is an ongoing need to provide alternative medical devices as well as alternative methods for manufacturing and using the medical devices.

### SUMMARY

This disclosure provides design, material, manufacturing method, and use alternatives for medical devices. An example medical device is an implantable heart valve configured for implantation within an existing prosthetic heart valve, comprising a frame comprising a plurality of frame struts defining a lattice region at an inflow end and a plurality of arches at an outflow end, and a central lumen extending therebetween, the plurality of frame struts in the lattice region defining a plurality of intersections and a plurality of open spaces, a plurality of protrusions extending radially outward from the plurality of frame struts, and a valve coupled to the frame, the valve positioned within the central lumen.

Alternatively or additionally to the embodiment above, the plurality of protrusions are disposed only on the lattice region of the frame.

Alternatively or additionally to any of the embodiments above, the lattice region is devoid of outwardly extending projections other than the plurality of protrusions.

Alternatively or additionally to any of the embodiments above, the valve includes a plurality of valve leaflets, wherein the plurality of valve leaflets are positioned between the lattice region and the plurality of arches.

Alternatively or additionally to any of the embodiments above, the plurality of frame struts in a region of the plurality of valve leaflets are devoid of the plurality of protrusions.

Alternatively or additionally to any of the embodiments above, each of the plurality of protrusions each have a length of between 1.5 mm and 5 mm.

Alternatively or additionally to any of the embodiments above, the length of each of the plurality of protrusions is between 2 mm and 3 mm.

Alternatively or additionally to any of the embodiments above, at least some of the plurality of protrusions extend substantially perpendicular to a longitudinal axis of the frame.

Alternatively or additionally to any of the embodiments above, at least some of the plurality of protrusions extend upward or downward at an angle of 35-55 degrees relative to a longitudinal axis of the frame.

Alternatively or additionally to any of the embodiments above, at least some of the plurality of protrusions extend upward or downward at an angle of 45 degrees relative to the longitudinal axis.

Alternatively or additionally to any of the embodiments above, the plurality of protrusions include a mixture of perpendicular protrusions and upwardly angled and downwardly angled protrusions.

Alternatively or additionally to any of the embodiments above, the plurality of protrusions are formed of a shape memory material, wherein the plurality of protrusions are aligned with an outer surface of the plurality of frame struts during delivery, and move to the radially outwardly extending position when deployed in a patient's body.

Alternatively or additionally to any of the embodiments above, the plurality of protrusions extend from at least some of the plurality of intersections.

Alternatively or additionally to any of the embodiments above, the plurality of open spaces in the lattice region are smaller than open spaces defined by the plurality of arches.

An example method of implanting a second heart valve within a previously implanted first heart valve comprises delivering the second heart valve to a site of the previously implanted first heart valve, the second heart valve including an expandable frame comprising a plurality of frame struts defining a lattice region at an inflow end and a plurality of arches at an outflow end, and a central lumen extending therebetween, the plurality of frame struts in the lattice region defining a plurality of intersections and a plurality of open spaces, a plurality of protrusions extending radially outward from the plurality of frame struts, and a valve coupled to the frame, the valve positioned within the central lumen. The method further includes inserting the second heart valve into the previously implanted first heart valve until the valve of the second heart valve is aligned coaxially with a valve region of the previously implanted first heart valve, and expanding the frame of the second heart valve.

Alternatively or additionally to the embodiment above, the plurality of protrusions are formed of a shape memory material, wherein when delivering the second heart valve, the plurality of protrusions are aligned with an outer surface of the plurality of frame struts, and after expanding the frame of the second heart valve, the plurality of protrusions move to the radially outwardly extending position.

Alternatively or additionally to any of the embodiments above, the plurality of protrusions are disposed only on the lattice region of the frame of the second heart valve, such that the lattice region of the second heart valve engages the first heart valve.

Alternatively or additionally to any of the embodiments above, the lattice region of the second heart valve is devoid of outwardly extending projections other than the plurality of protrusions.

Alternatively or additionally to any of the embodiments above, the plurality of protrusions have a length of between 2 mm and 3 mm, and extend upward or downward at an angle of 35-55 degrees relative to a longitudinal axis of the frame.

Another example implantable heart valve assembly comprises a first implantable heart valve including a frame comprising a plurality of frame struts defining a lattice region at an inflow end of the frame and a plurality of arches at an outflow end of the frame, and a central lumen extending therebetween, the lattice region having a first end defining the inflow end of the frame and a second end adjacent the plurality of arches, wherein at least some of the plurality of frame struts at the second end define outwardly extending stent regions, and a valve coupled to the frame, the valve positioned within the central lumen, and a second implantable heart valve including a frame comprising a plurality of frame struts defining a lattice region at an inflow end and a plurality of arches at an outflow end, and a central lumen extending therebetween, the plurality of frame struts in the lattice region defining a plurality of intersections and a plurality of open spaces, the second implantable heart valve including a plurality of protrusions extending radially outward from the plurality of frame struts, and a valve coupled to the frame, the valve positioned within the central lumen.

The above summary of some embodiments, aspects, and/or examples is not intended to describe each embodiment or every implementation of the present disclosure. The figures and the detailed description which follows more particularly exemplify these embodiments.

### BRIEF DESCRIPTION OF THE DRAWINGS

The disclosure may be more completely understood in consideration of the following detailed description of various embodiments in connection with the accompanying drawings, in which:

FIG. 1 illustrates a prior art prosthetic heart valve;

FIG. 2 illustrates the prosthetic heart valve of FIG. 1 implanted within another prosthetic heart valve of FIG. 1, with valve leaflets omitted;

FIG. 3 is a schematic side view of an example prosthetic heart valve according to one embodiment of the invention, showing an interface between a frame and valve leaflets;

FIG. 4 illustrates the prosthetic heart valve of FIG. 3 implanted within the prosthetic heart valve of FIG. 1, with valve leaflets omitted;

FIGS. 5A-5C illustrate an example foldable protrusion; and

FIGS. 6 and 7 illustrate example orientations of the foldable protrusions of FIGS. 5A-5C on a prosthetic heart valve according to the invention, with valve leaflets omitted.

While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5). Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

Relative terms such as “proximal”, “distal”, “advance”, “withdraw”, variants thereof, and the like, may be generally considered with respect to the positioning, direction, and/or operation of various elements relative to a user/operator/manipulator of the device, wherein “proximal” and “withdraw” indicate or refer to closer to or toward the user and “distal” and “advance” indicate or refer to farther from or away from the user. In some instances, the terms “proximal” and “distal” may be arbitrarily assigned in an effort to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device.

The term “extent” may be understood to mean a greatest measurement of a stated or identified dimension, unless the extent or dimension in question is preceded by or identified as a “minimum”, which may be understood to mean a smallest measurement of the stated or identified dimension. For example, “outer extent” may be understood to mean a maximum outer dimension, “radial extent” may be understood to mean a maximum radial dimension, “longitudinal extent” may be understood to mean a maximum longitudinal dimension, etc. Each instance of an “extent” may be different (e.g., axial, longitudinal, lateral, radial, circumferential, etc.) and will be apparent to the skilled person from the context of the individual usage. Generally, an “extent” may be considered a greatest possible dimension measured according to the intended usage, while a “minimum extent” may be considered a smallest possible dimension measured according to the intended usage. In some instances, an “extent” may generally be measured orthogonally within a plane and/or cross-section, but may be, as will be apparent from the particular context, measured differently—such as, but not limited to, angularly, radially, circumferentially (e.g., along an arc), etc.

The terms “monolithic” and “unitary” shall generally refer to an element or elements made from or consisting of a single structure or base unit/element. A monolithic and/or unitary element shall exclude structure and/or features made by assembling or otherwise joining multiple discrete elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to affect the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously-used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

The following description should be read with reference to the drawings, which are not necessarily to scale, wherein similar elements in different drawings are numbered the same. The detailed description and drawings are intended to illustrate but not limit the disclosure. Those skilled in the art will recognize that the various elements described and/or shown may be arranged in various combinations and configurations without departing from the scope of the disclosure. The detailed description and drawings illustrate example embodiments of the disclosure. However, in the interest of clarity and ease of understanding, while every feature and/or element may not be shown in each drawing, the feature(s) and/or element(s) may be understood to be present regardless, unless otherwise specified.

As TAVI moves into lower risk patients, a growing need may arise for replacing a first prosthetic valve with a second prosthetic valve. Valve-in-valve (VIV) has become a feasible alternative to surgical replacement for treating patients who have degenerated prosthetic aortic valves.

However, implanting a second prosthetic heart valve directly within a previously-implanted prosthetic heart valve may be impractical because the second prosthetic heart valve (including the support structure and valve assembly) will have to reside within the annulus of the previously-implanted heart valve. This may reduce the effective orifice area (EOA) available for blood flow because traditional prosthetic heart valves may not be configured to easily receive another prosthetic valve in a manner which provides secure seating for the new valve while also having a large enough annulus within the new valve to provide the desired EOA.

In addition to the space taken up by degenerated leaflets and accumulated material in the failed prosthetic valve, the support structure and valve leaflets in the second prosthetic heart valve may contribute to a reduced EOA. Obtaining an adequate (EOA) in valve replacement may minimize pressure gradients across the prosthetic aortic valve and improve clinical outcomes. Reducing the outer diameter of the second prosthetic valve, and/or reducing the distance between the outer diameter and inner diameter of the second prosthetic valve, may achieve the desired EOA in a valve-in-valve procedure.

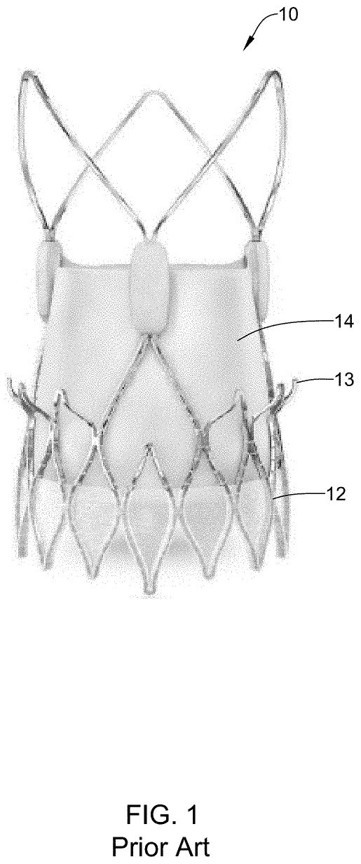

As shown in FIG. 1, prior art prosthetic heart valves 10 may include valve leaflets 14 attached to an expandable stent structure 12 including outwardly protruding stent regions 13 designed to engage the native valve leaflets to prevent migration of the prosthetic heart valve 10. The outwardly protruding stent regions 13 may be formed by bending and heat setting upper portions of some stent regions of the stent structure 12.

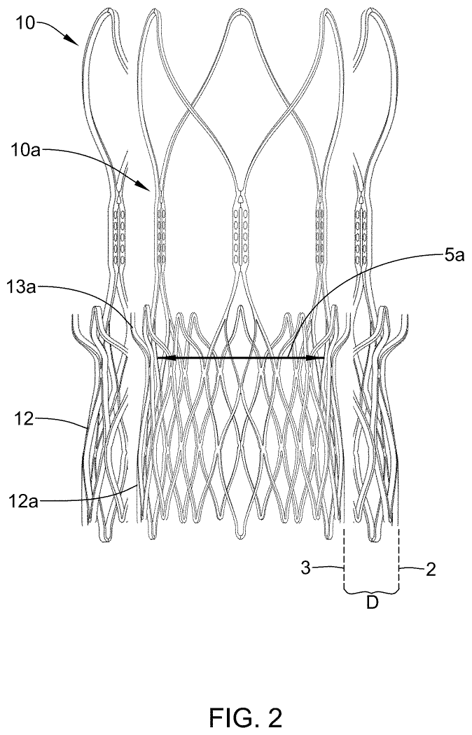

FIG. 2 illustrates how the EOA may be reduced if a second prosthetic heart valve 10a having outwardly protruding stent regions 13a is inserted within a first valve 10 having the same structure. The valve leaflets of both valves 10, 10a are not shown for clarity. The outwardly protruding stent regions 13a on the inner or second prosthetic heart valve 10a may engage the inner surface of the stent structure 12 of the first prosthetic heart valve 10 and may cause the stent structure 12a of the second prosthetic heart valve 10a to be spaced apart from the expandable stent structure 12 of the first prosthetic heart valve 10. The outwardly protruding stent regions 13a create a distance D between the outermost diameter 2 of the first stent structure 12 and the inner diameter 3 of the second stent structure 12a. This distance D may prevent the second prosthetic heart valve 10a from fully expanding and may reduce the inner diameter (arrow 5a) of the second prosthetic heart valve 10a. The reduced inner diameter (arrow 5a) of the second prosthetic heart valve 10a may reduce the EOA, the geometric orifice area (GOA), and blood flow. The prosthetic valve design including outwardly protruding stent regions 13 as in FIGS. 1 and 2 may require a higher placement of the second prosthetic heart valve 10a to avoid impingement of the outwardly protruding stent regions 13a reducing the EOA. However, this approach may not be a satisfactory solution due to the risk of reducing blood flow and undesirable location of the prosthetic valve.

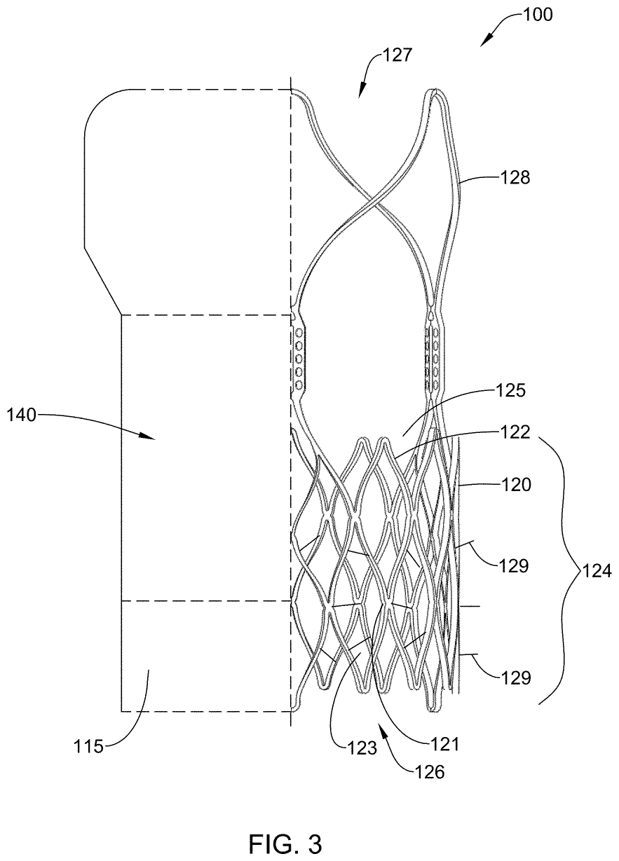

FIG. 3 illustrates an example prosthetic heart valve 100 that, when implanted within a previously-implanted prosthetic heart valve, may maintain a desired EOA. The heart valve 100 may include an expandable frame 120 comprising a plurality of frame struts 122 defining a lattice region 124 at an inflow end 126 and a plurality of arches 128 at an outflow end 127, and a central lumen 125 extending therebetween. The frame struts 122 in the lattice region 124 may define a plurality of intersections 121 and a plurality of open spaces 123. As shown in FIG. 3, the open spaces 123 in the lattice region 124 may be smaller than open spaces defined by the arches 128. The frame 120 may include a plurality of valve leaflets 140 coupled to the frame to define a valve, with the valve positioned within the central lumen 125. The position of the valve leaflets 140 is depicted schematically by the bounding phantom lines. The plurality of valve leaflets 140 may be positioned within the lattice region 124 and below the plurality of arches 128.

The prosthetic heart valve 100 does not have the outwardly protruding stent regions 13 designed to engage the native valve leaflets, as shown in the heart valve 10 in FIG. 1. The absence of the outwardly extending stent regions allows the heart valve 100 to be expanded into close contact with the frame of the previously-implanted valve 10.

In some embodiments, the frame 120 may include a plurality of small protrusions 129 extending radially outward from the frame struts 122. The protrusions 129 may provide point loads for engaging the stent structure 12 of the previously-implanted valve 10 when the prosthetic heart valve 100 is inserted into the previously-implanted valve 10. In some embodiments, the plurality of protrusions 129 may be disposed on the entire lattice region 124. In other embodiments, the plurality of protrusions 129 may be disposed only on the lower portion 115 of the lattice region 124, below the valve leaflets 140, leaving the frame struts 122 in the region of the valve leaflets 140 devoid of the plurality of protrusions 129. The lattice region 124 may be devoid of any outwardly extending projections other than the plurality of protrusions 129. For example, the lattice region 124 may be devoid of any struts bent and heat set into radially outward projections.

The plurality of protrusions 129 may have a length of between 1.5 mm and 5 mm. In some embodiments, the plurality of protrusions 129 may have a length of between 2 mm and 3 mm. The protrusions 129 may be linear along an entirety of their length. In some embodiments, at least some of the protrusions 129 may be curved along at least a portion of their length to define a hook. In other embodiments, at least some of the protrusions 129 may have a linear region extending from the strut 122 and a curved or hooked free end. The plurality of protrusions 129 may extend substantially perpendicular to a longitudinal axis of the frame 120, or the protrusions 129 may extend at an angle relative to the longitudinal axis. In some embodiments, the protrusions 129 may extend upward or downward at an angle of 35-55 degrees relative to the longitudinal axis of the frame 120. For example, the plurality of protrusions 129 may extend upward or downward at an angle of 45 degrees relative to the longitudinal axis. The plurality of protrusions 129 may all extend at the same angle, or the plurality of protrusions 129 may include a mixture of both upwardly angled and downwardly angled protrusions as well as perpendicular protrusions, as shown in FIG. 3. The protrusions 129 may prevent upwards or downwards movement of the frame 120 relative to the previously-implanted valve 10, to better anchor the second prosthetic heart valve 100 after valve-in-valve implantation. Preventing relative movement of the frame 120 may also prevent embolization in the region.

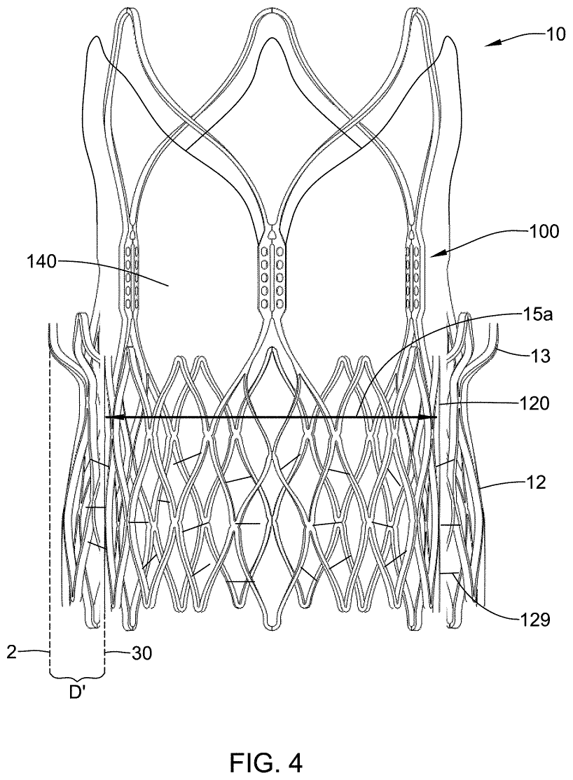

The lack of the outwardly protruding stent regions 13 in the region of the valve leaflets 140 may allow the second or inner prosthetic heart valve 100 to be inserted into and fully expanded within the previously-implanted valve 10, while maintaining a desired inner diameter (arrow 15a) only slightly smaller than the inner diameter of the previously-implanted valve 10, as shown in FIG. 4. The valve leaflets of the previously-implanted valve 10 and the second prosthetic heart valve 100 are not shown for clarity. The lack of outwardly extending stent regions creates a significantly smaller distance D′ between the outermost diameter 2 of the first stent structure 12 (based on the outwardly protruding stent regions 13) and the inner diameter 30 of the frame 120 of the second prosthetic heart valve 100. A valve-in-valve structure of the second prosthetic heart valve 100 within a previously-implanted valve 10 maintains a desired EOA.

In some embodiments, the struts 122 may be cut from a nitinol tube to form the frame 120. The plurality of protrusions 129 may also be cut from the nitinol tube or from the struts 122, such that the plurality of protrusions 129 are aligned with an outer surface of the struts 122 during delivery when the frame 120 is constrained within a delivery sheath, and move to the radially outwardly extending position when released from the delivery sheath and deployed in a patient's body. Additionally, the plurality of protrusions 129 may be cut from the nitinol tube to extend from at least some of the intersections 121. The protrusions 129 may be thinner than the frame struts 122. In some embodiments, the protrusions 129 may have a thickness that is 50% of the thickness of the frame struts 122. In other embodiments, the protrusions 129 may be thin nitinol or polymeric rods attached to the frame 120.

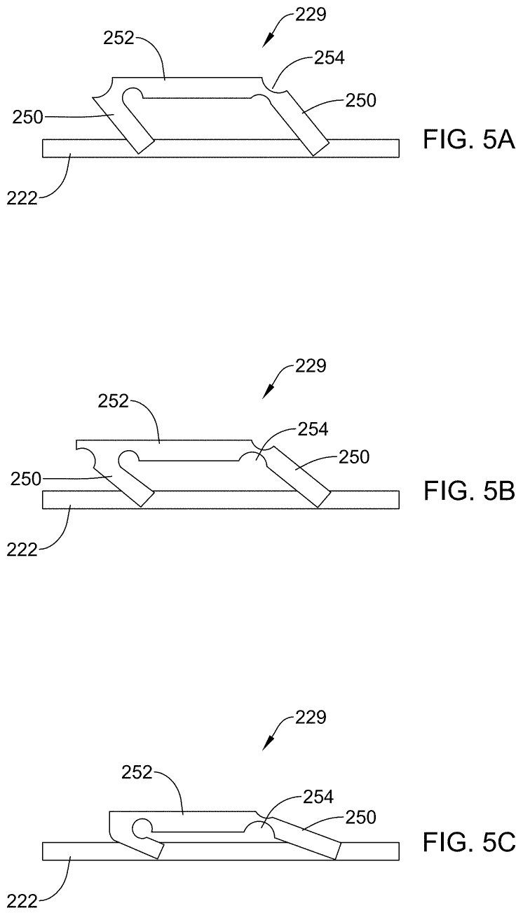

In another embodiment, the plurality of protrusions may be foldable protrusions 229, as illustrated in FIGS. 5A-5C. Each foldable protrusion 229 may include first and second legs 250 coupled with a top section 252. The legs 250 and top section 252 may be formed from a single elongate rod, with material removed at cutouts 254 forming hinges at the junctions between the legs 250 and top section 252. The legs 250 may each be flexibly attached to a strut 222, for example with a suture or flexible adhesive. The top section 252 protrudes from the stent, forming a point load when the stent is deployed. The cutouts 254 allow the foldable protrusions 229 to shift from a biased upright configuration, as shown in FIG. 5A, to an intermediate folding configuration, as shown in FIG. 5B, and to a folded configuration as shown in FIG. 5C. When the stent is constrained in a sheath for delivery, the foldable protrusions 229 may be folded against the stent frame, in the folded configuration as shown in FIG. 5C. When the sheath is withdrawn and the stent expands, the foldable protrusions 229 may expand to their biased upright configuration as shown in FIG. 5A. The foldable protrusions 229 may be formed from a shape-memory material such as nitinol or a shape-memory polymer. In other examples, the foldable protrusions 229 may be formed as a monolithic structure with a bottom rod in place of the strut 222 shown in FIGS. 5A-5C. The monolithic structure may be cut from a sheet, with material removed at cutouts 254 at each corner of the formed rectangle, allowing the legs 250 to move as shown in FIGS. 5B and 5C as the foldable protrusion 229 folds against the bottom strut 222.

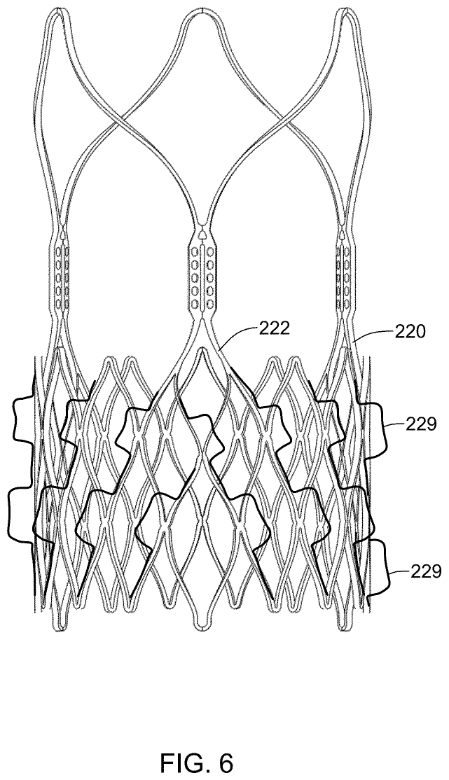

The foldable protrusions 229 may be attached to the stent frame 220, extending along the struts 222, as shown in FIG. 6. The foldable protrusions 229 may be attached to the struts 222 with sutures or flexible adhesive. The foldable protrusions 229 follow the angle of the struts 222. In another embodiment, the foldable protrusions 229 may be cut from the strut 222, with one of the legs 250 remaining attached to the strut 222 and the opposite leg 250 forming a free end. The free end may be attached to the strut 222 with a suture or flexible adhesive.

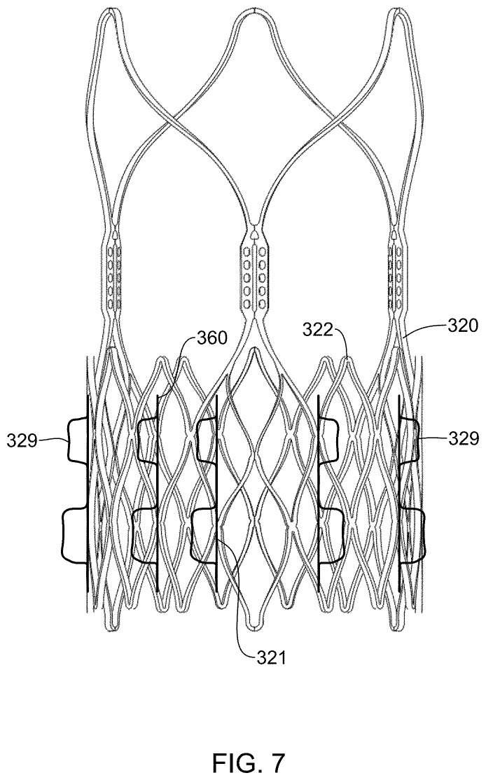

In another embodiment, the foldable protrusions 329 may be attached to separate rods 360 which are then attached to the stent frame 320, as shown in FIG. 7. In this embodiment, the rods 260 and foldable protrusions 329 may extend axially along the frame 320, instead of following the angle of the struts 322. The rods 360 may be attached to the struts 322 at intersections 321 or along the length of the struts 322. The rods 360 may be made of a shape-memory material such as nitinol or polymer, and may be attached to the frame 320 with sutures or adhesive.

In further embodiments, the foldable protrusions 229 attached along the struts 222 or the foldable protrusions 329 attached via separate rods 360, may be formed from shape-memory polymer or shape-memory metal elongate members, without the cutouts shown in FIGS. 5A-5C, with the flexibility of the shape-memory metal or polymer causing the protrusions 229, 329 to be biased in an outward projecting arch, and foldable against the stent frame when the stent is constrained for delivery. The shape and position of such protrusions 229, 329 may be the same as that shown in FIGS. 6 and 7. The foldable protrusions 229 may have a length of 5-10 mm, measured along the top section 252. The legs 250 may have a length of 2-5 mm.

A further embodiment may include a mixture of protrusions 129 and foldable protrusions 229, and some of the protrusions 129, 229 may be formed from the stent frame 120, 220 and other protrusions 129, 229 may be attached directly to the stent frame 120, 220 or attached to separate rods which may then be attached to the stent frame.

A method of implanting the prosthetic heart valve 100 as a second valve within a previously implanted first heart valve may include delivering the second heart valve 100 to the site of the previously implanted first prosthetic heart valve 10, inserting the second heart valve 100 into the previously implanted first heart valve 10 until the valve 140 of the second heart valve 100 is aligned coaxially with a valve of the previously implanted first prosthetic heart valve 10. Once the second heart valve 100 is in position, it may be expanded to engage the protrusions 129 on the second heart valve 100 with the stent structure 12 of the first valve 10. Expansion of the second heart valve 100 may be achieved with an expandable element such as a balloon. In other embodiments, the frame 120 of the second valve may be self-expanding. When at least the protrusions 129 are formed of a shape memory material, the protrusions may move from a first position aligned with an outer surface of the struts 122 during delivery, to a second, radially outwardly extended position when positioned within the body. The frame 120 and protrusions 129 may be cut from a shape memory material tube and heat set to 27° C. such that valve assembly is not impacted yet the protrusions 129 will move to the radially extended position at body temperature (37° C.).

The prosthetic heart valve 100 may allow valve-in-valve procedures to be performed on previously-implanted valves 10 with outwardly protruding stent regions 13, without a significant reduction in effective orifice area (EOA). In some embodiments, the prosthetic heart valve 100 may be made in a similar manner as the heart valve 10, but without the step of heat setting the outwardly protruding stent regions 13.

An implantable heart valve assembly or kit for performing a valve-in-valve implantation procedure may include a first implantable heart valve 10 including a stent structure 12 comprising a plurality of frame struts defining a lattice region at an inflow end of the frame and a plurality of arches at an outflow end of the frame, and a central lumen extending therebetween. The lattice region of the first valve 10 may have a first end defining the inflow end of the frame and a second end adjacent the plurality of arches, where at least some of the frame struts at the second end define outwardly protruding stent regions 13, and a valve may be coupled to the frame, positioned within the central lumen. The assembly or kit may also include a second prosthetic heart valve 100 configured to be implanted within the first valve 10. The second heart valve 100 may include a frame 120 including a plurality of frame struts 122 defining a lattice region 124 at an inflow end 126 and a plurality of arches 128 at an outflow end 127, and a central lumen 125 extending therebetween. The frame struts 122 in the lattice region 124 may define a plurality of intersections 121 and a plurality of open spaces 123. The second prosthetic heart valve 100 may include a plurality of protrusions 129 extending radially outward from the frame struts 122, and a valve 140 coupled to the frame 120, where the valve 140 is positioned within the central lumen 125. In some embodiments, the second prosthetic heart valve 100 may be sized the same as the first valve 10.

The materials that can be used for the various components of the prosthetic heart valve 10, 100 (and/or other systems or components disclosed herein) and the various elements thereof disclosed herein may include those commonly associated with medical devices. For simplicity purposes, the following discussion makes reference to the heart valve 100 (and variations, systems or components disclosed herein). However, this is not intended to limit the devices and methods described herein, as the discussion may be applied to other elements, members, components, or devices disclosed herein.

In some embodiments, heart valve 100 (and variations, systems or components thereof disclosed herein) may be made from a metal, metal alloy, ceramics, zirconia, polymer (some examples of which are disclosed below), a metal-polymer composite, combinations thereof, and the like, or other suitable material. Some examples of suitable metals and metal alloys include stainless steel, such as 444V, 444L, and 314LV stainless steel; mild steel; nickel-titanium alloy such as linear-elastic and/or super-elastic nitinol; cobalt chromium alloys, titanium and its alloys, alumina, metals with diamond-like coatings (DLC) or titanium nitride coatings, other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R44035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R44003 such as ELGILOY®, PHYNOX®, and the like); platinum enriched stainless steel; titanium; platinum; palladium; gold; combinations thereof; and the like; or any other suitable material.

As alluded to herein, within the family of commercially available nickel-titanium or nitinol alloys, is a category designated “linear elastic” or “non-super-elastic” which, although may be similar in chemistry to conventional shape memory and super-elastic varieties, may exhibit distinct and useful mechanical properties. Linear elastic and/or non-super-elastic nitinol may be distinguished from super-elastic nitinol in that the linear elastic and/or non-super-elastic nitinol does not display a substantial “super-elastic plateau” or “flag region” in its stress/strain curve like super-elastic nitinol does. Instead, in the linear elastic and/or non-super-elastic nitinol, as recoverable strain increases, the stress continues to increase in a substantially linear, or a somewhat, but not necessarily entirely linear relationship until plastic deformation begins or at least in a relationship that is more linear than the super-elastic plateau and/or flag region that may be seen with super-elastic nitinol. Thus, for the purposes of this disclosure linear elastic and/or non-super-elastic nitinol may also be termed “substantially” linear elastic and/or non-super-elastic nitinol.

In some cases, linear elastic and/or non-super-elastic nitinol may also be distinguishable from super-elastic nitinol in that linear elastic and/or non-super-elastic nitinol may accept up to about 2-5% strain while remaining substantially elastic (e.g., before plastically deforming) whereas super-elastic nitinol may accept up to about 8% strain before plastically deforming. Both of these materials can be distinguished from other linear elastic materials such as stainless steel (that can also be distinguished based on its composition), which may accept only about 0.2 to 0.44 percent strain before plastically deforming.

In some embodiments, the linear elastic and/or non-super-elastic nickel-titanium alloy is an alloy that does not show any martensite/austenite phase changes that are detectable by differential scanning calorimetry (DSC) and dynamic metal thermal analysis (DMTA) analysis over a large temperature range. For example, in some embodiments, there may be no martensite/austenite phase changes detectable by DSC and DMTA analysis in the range of about −60 degrees Celsius (° C.) to about 120° C. in the linear elastic and/or non-super-elastic nickel-titanium alloy. The mechanical bending properties of such material may therefore be generally inert to the effect of temperature over this very broad range of temperature. In some embodiments, the mechanical bending properties of the linear elastic and/or non-super-elastic nickel-titanium alloy at ambient or room temperature are substantially the same as the mechanical properties at body temperature, for example, in that they do not display a super-elastic plateau and/or flag region. For example, across a broad temperature range, the linear elastic and/or non-super-elastic nickel-titanium alloy maintains its linear elastic and/or non-super-elastic characteristics and/or properties.

In some embodiments, the linear elastic and/or non-super-elastic nickel-titanium alloy may be in the range of about 50 to about 60 weight percent nickel, with the remainder being essentially titanium. In some embodiments, the composition is in the range of about 54 to about 57 weight percent nickel. One example of a suitable nickel-titanium alloy is FHP-NT alloy commercially available from Furukawa Techno Material Co. of Kanagawa, Japan. Other suitable materials may include ULTANIUM™ (available from Neo-Metrics) and GUM METAL™ (available from Toyota). In some other embodiments, a super-elastic alloy, for example a super-elastic nitinol can be used to achieve desired properties.

In at least some embodiments, portions or all of the heart valve 100 (and variations, systems or components thereof disclosed herein) may also be doped with, made of, or otherwise include a radiopaque material. Radiopaque materials are understood to be materials capable of producing a relatively bright image on a fluoroscopy screen or another imaging technique during a medical procedure. This relatively bright image aids a user in determining the location of the heart valve 100 (and variations, systems or components thereof disclosed herein). Some examples of radiopaque materials can include, but are not limited to, gold, platinum, palladium, tantalum, tungsten alloy, polymer material loaded with a radiopaque filler, and the like. Additionally, other radiopaque marker bands may also be incorporated into the design of the heart valve 100 (and variations, systems or components thereof disclosed herein) to achieve the same result.

In some embodiments, the heart valve 100 (and variations, systems or components thereof disclosed herein) and/or portions thereof, may be made from or include a polymer or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex® high-density polyethylene, Marlex® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, polyurethane silicone copolymers (for example, Elast-Eon® from AorTech Biomaterials or ChronoSil® from AdvanSource Biomaterials), biocompatible polymers, other suitable materials, or mixtures, combinations, copolymers thereof, polymer/metal composites, and the like. In some embodiments, the sheath can be blended with a liquid crystal polymer (LCP). For example, the mixture can contain up to about 6 percent LCP.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The disclosure's scope is, of course, defined in the language in which the appended claims are expressed.

What is claimed is:

1. An implantable heart valve configured for implantation within an existing prosthetic heart valve, comprising:
a frame comprising a plurality of frame struts defining a lattice region at an inflow end and a plurality of arches at an outflow end, and a central lumen extending therebetween, the plurality of frame struts in the lattice region defining a plurality of intersections and a plurality of open spaces;
a plurality of protrusions extending radially outward from the plurality of frame struts; and
a valve coupled to the frame, the valve positioned within the central lumen.

2. The implantable heart valve of claim 1, wherein the plurality of protrusions are disposed only on the lattice region of the frame.

3. The implantable heart valve of claim 2, wherein the lattice region is devoid of outwardly extending projections other than the plurality of protrusions.

4. The implantable heart valve of claim 1, wherein the valve includes a plurality of valve leaflets, wherein the plurality of valve leaflets are positioned between the lattice region and the plurality of arches.

5. The implantable heart valve of claim 4, wherein the plurality of frame struts in a region of the plurality of valve leaflets are devoid of the plurality of protrusions.

6. The implantable heart valve of claim 1, wherein each of the plurality of protrusions each have a length of between 1.5 mm and 5 mm.

7. The implantable heart valve of claim 6, wherein the length of each of the plurality of protrusions is between 2 mm and 3 mm.

8. The implantable heart valve of claim 1, wherein at least some of the plurality of protrusions extend substantially perpendicular to a longitudinal axis of the frame.

9. The implantable heart valve of claim 1, wherein at least some of the plurality of protrusions extend upward or downward at an angle of 35-55 degrees relative to a longitudinal axis of the frame.

10. The implantable heart valve of claim 9, wherein at least some of the plurality of protrusions extend upward or downward at an angle of 45 degrees relative to the longitudinal axis.

11. The implantable heart valve of claim 8, wherein the plurality of protrusions include a mixture of perpendicular protrusions and upwardly angled and downwardly angled protrusions.

12. The implantable heart valve of claim 1, wherein the plurality of protrusions are formed of a shape memory material, wherein the plurality of protrusions are aligned with an outer surface of the plurality of frame struts during delivery, and move to the radially outwardly extending position when deployed in a patient's body.

13. The implantable heart valve of claim 1, wherein the plurality of protrusions extend from at least some of the plurality of intersections.

14. The implantable heart valve of claim 1, wherein the plurality of open spaces in the lattice region are smaller than open spaces defined by the plurality of arches.

15. A method of implanting a second heart valve within a previously implanted first heart valve, comprising:
delivering the second heart valve to a site of the previously implanted first heart valve, the second heart valve including:
an expandable frame comprising a plurality of frame struts defining a lattice region at an inflow end and a plurality of arches at an outflow end, and a central lumen extending therebetween, the plurality of frame struts in the lattice region defining a plurality of intersections and a plurality of open spaces;
a plurality of protrusions extending radially outward from the plurality of frame struts; and
a valve coupled to the frame, the valve positioned within the central lumen;
inserting the second heart valve into the previously implanted first heart valve until the valve of the second heart valve is aligned coaxially with a valve region of the previously implanted first heart valve; and
expanding the frame of the second heart valve.

16. The method of claim 15, wherein the plurality of protrusions are formed of a shape memory material, wherein when delivering the second heart valve, the plurality of protrusions are aligned with an outer surface of the plurality of frame struts, and after expanding the frame of the second heart valve, the plurality of protrusions move to the radially outwardly extending position.

17. The method of claim 15, wherein the plurality of protrusions are disposed only on the lattice region of the frame of the second heart valve, such that the lattice region of the second heart valve engages the first heart valve.

18. The method of claim 17, wherein the lattice region of the second heart valve is devoid of outwardly extending projections other than the plurality of protrusions.

19. The method of claim 15, wherein the plurality of protrusions have a length of between 2 mm and 3 mm, and extend upward or downward at an angle of 35-55 degrees relative to a longitudinal axis of the frame.

20. An implantable heart valve assembly, comprising:
a first implantable heart valve including a frame comprising a plurality of frame struts defining a lattice region at an inflow end of the frame and a plurality of arches at an outflow end of the frame, and a central lumen extending therebetween, the lattice region having a first end defining the inflow end of the frame and a second end adjacent the plurality of arches, wherein at least some of the plurality of frame struts at the second end define outwardly extending stent regions, and a valve coupled to the frame, the valve positioned within the central lumen; and
a second implantable heart valve including a frame comprising a plurality of frame struts defining a lattice region at an inflow end and a plurality of arches at an outflow end, and a central lumen extending therebetween, the plurality of frame struts in the lattice region defining a plurality of intersections and a plurality of open spaces, the second implantable heart valve including a plurality of protrusions extending radially outward from the plurality of frame struts, and a valve coupled to the frame, the valve positioned within the central lumen.
